(19) United States
(12) Patent Application Publication
(10) Pub. No.: US 2025/0375374 A1
(43) Pub. Date: Dec. 11, 2025

(54) SHEAR-THINNING COMPOSITIONS FOR ABLATION

(71) Applicant: Boston Scientific Scimed, Inc., Maple Grove, MN (US)

(72) Inventors: Ehsan Jabbarzadeh, Columbia, SC (US); Sara Eslambolchi Moghadam, Santa Clara, CA (US); Karen Dubbin, Mountain View, CA (US)

(73) Assignee: Boston Scientific Scimed, Inc., Maple Grove, MN (US)

(21) Appl. No.: 18/649,744

(22) PCT Filed: Jul. 28, 2022
(86) PCT No.: PCT/US2022/038678

§ 371 Date: Apr. 29, 2024

Related U.S. Application Data
(60) Provisional application No. 63/249,764, filed on Sep. 29, 2021.

(51) Int. Cl. A61K 9/06; A61K 9/00; A61K 31/045; A61K 45/06; A61K 47/02; A61K 49/00

(52) U.S. Cl. CPC A61K 9/06; A61K 9/0024; A61K 31/045; A61K 45/06; A61K 47/02; A61K 49/0002

(57) ABSTRACT

Provided herein are shear-thinning compositions for use e.g., in ablation. neurolysis, or sclerotherapy.

### CROSS-REFERENCE TO RELATED APPLICATIONS

This application claims priority to PCT International Application No. PCT/US2022/038678, filed on Jul. 28, 2022, entitled “SHEAR-THINNING COMPOSITIONS FOR ABLATION”, which claims the benefit of U.S. Provisional Patent Application No. 63/249,764, filed on Sep. 29, 2021, the contents of which are herein incorporated by reference in their entirety.

### STATEMENT REGARDING FEDERALLY SPONSORED RESEARCH

This invention was made with government support under 1R44HL158397-01 awarded by National Institutes of Health. The government has certain rights in the invention.

### BACKGROUND

Sclerotherapy has traditionally been used to destroy encapsulated structures. Low- or medium-viscosity liquid agents can coat an entire cyst or vascular cell lining or penetrate an entire tumor structure. Limitations to efficacy thus principally result from failure of penetration or faster agent clearance and decay rates relative to destructive effects, and often lead to a partial response and repeat sclerosis. Techniques have also been hampered by nonselective effects sometimes leading to painful and irritating local tissue destruction, non-target effects, and systemic absorption related side effects, such as pulmonary collapse.

Ethanol is the standard sclerotherapy agent and has been used in a variety of settings, including vascular, pleural, tumoral, and cyst disorders. Its mechanism of action is a combination of cytotoxic damage induced by the denaturation and extraction of surface proteins, hypertonic dehydration of cells, and coagulation and thrombosis when blood products are present. All these factors lead to fibrinoid necrosis. Ethanol's deep penetration into the vascular wall and lack of viscosity allows it to target to most tissues, although its efficacy is limited by, for example, leakage into nearby tissue and non-target vessel necrosis. Additionally, effectiveness of the treatment requires multiple sessions and injection of large volumes of ethanol which risks damage to surrounding tissue.

Neurolysis is a technique used in pain management where neurolytic agents are used to induce temporary degeneration of nerve fibers related to transmitting pain. To this end, chemical neurolytics, including ethanol and phenol, can be delivered to a group of nerves, called a plexus or a ganglion, to block the pain to a specific organ or region of the body. Such technique is used when disease treatment or other forms of pain management have proved ineffective. Nerve blocks achieved through neurolysis have been used to prevent opioid escalation in conditions including inoperable cancer, arterial occlusive disease, and peripheral neuralgia. This conscious nerve damage is used as a nerve block to cause semi-enduring block of sensory function resulting in pain relief for palliative care. Some common nerve blocks using neurolysis includes blocks of the celiac or brachial plexus.

Ethanol is a commonly used neurolytic agent in neurolysis applications as it is considered to cause efficient neural destruction, rapid onset of action, and has demonstrated an ability to mix with a contrast agent. Ethanol treatment can lead to nerve blocks lasting 3-6 months. Current applications of this technique involve injections of large volumes of ethanol to the nerve ganglion of interest due to low viscosity and high dispersity of alcohol in tissue. Because this large volume of ethanol injection is also associated with patient pain, the development of a localized method of neurolysis with longer retention is needed.

At least in view of the above, there exist a need for new compositions that allow for controlled release of sclerosing, neurolytic, and other therapies for a variety of applications.

### SUMMARY

In one aspect, the present disclosure provides a composition for use in ablation or sclerotherapy, comprising an effective amount of ethanol or a derivative thereof, silicate nanoparticles, and water, wherein the composition has a viscosity of about 2,000-15,000 mPa·s.

In some embodiments, the ethanol is absolute ethanol.

In some embodiments, upon injection of compositions of the present disclosure to a patient in need thereof, the implant stays in place for at least 90 days.

In some embodiments, upon injection of compositions of the present disclosure to a patient in need thereof, the implant elutes the ethanol or a derivative thereof for at least 90 days.

In some embodiments, the composition comprises about 5% to about 15% by weight of silicate nanoparticles. In some embodiments, the composition comprises about 6% to about 15% by weight of silicate nanoparticles, or about 8% to about 15% by weight of silicate nanoparticles, or about 10% to about 15% by weight of silicate nanoparticles.

In some embodiments, the ratio of water to ethanol or a derivative thereof in compositions of the present disclosure is from about 100:0 to about 50:50 by weight.

In some embodiments, the ratio of silicate nanoparticles to ethanol or a derivative thereof in compositions of the present disclosure is about 75:25 by weight.

In some embodiments, the ratio of silicate nanoparticles to ethanol or a derivative thereof in compositions of the present disclosure is about 60:40 by weight.

In some embodiments, the composition further comprises a contrast agent.

In some embodiments, the composition further comprises a therapeutic agent selected from the group consisting of chemotherapeutic agents and sclerosing agents.

In one aspect, the present disclosure provides methods of treating a lesion, comprising administering a therapeutically effective amount of a composition of the present disclosure to a patient in need thereof.

In some embodiments, the present disclosure provides a method of sclerotherapy, comprising administering a therapeutically effective amount of a composition of the present disclosure to a patient in need thereof.

According to an embodiment, the present disclosure describes a composition for use in neurolysis, comprising an effective amount of a neurolytic agent, such as ethanol or phenol, silicate nanoparticles, and water.

In an embodiment, the composition for use in neurolysis comprises one or more polymers including at least one of poly(N-isopropylacrylamide) (PNIPAM), poly(lactic-co-glycolic acid) (PLGA), poly(lactic acid) (PLA), polyethylene glycol (PEG), poly(vinyl alcohol) (PVA), gelatin, chitosan, or collagen.

In an embodiment, upon injection to a patient in need thereof, the composition for use in neurolysis elutes the neurolytic agent.

In an embodiment, the composition for use in neurolysis includes contrast agent.

In an embodiment, the present disclosure describes a method of pain management, comprising administering a therapeutically effective amount of the composition for use in neurolysis.

In an embodiment, the method of pain management is for palliative care in cancer treatment.

In an embodiment, the method of pain management is for ischemic rest pain.

In an embodiment, the composition for use in neurolysis is used for renal denervation.

In an embodiment, the composition for use in neurolysis is used for genicular nerve degeneration.

According to an embodiment, the present disclosure describes a composition for ablation of residual tumor cells post-surgical resection, comprising an effective amount of an ablative agent, such as ethanol, silicate nanoparticles, and water.

In an embodiment, the composition for ablation is delivered via spraying.

In an embodiment, the composition for ablation further comprises a contrast agent.

In an embodiment, the composition further comprises one or more polymers including at least one of PNIPAM, PLGA, PLA, PVA, gelatin, chitosan, and collagen.

In an embodiment, the present disclosure describes a method of tumor therapy, comprising administering a therapeutically effective amount of the composition for ablation to a patient in need thereof.

In an embodiment, the tumor therapy is applied following full surgical resection.

In an embodiment, the tumor therapy is applied proactively at the time of full surgical resection.

In an embodiment, the tumor therapy is applied in the case of positive surgical margins.

In an embodiment, the composition ablates tumor margins.

In an embodiment, the tumor therapy is applied proactively at the time of debulking.

In an embodiment, the composition for ablation further serves as a space-filling material after surgical resection or debulking.

In an embodiment, the composition further comprises a therapeutic agent selected from the group consisting of chemotherapeutic agents and immune oncology agents.

### BRIEF DESCRIPTIONS OF THE FIGURES

FIG. 1 depicts photographs of shear-thinning composition A.

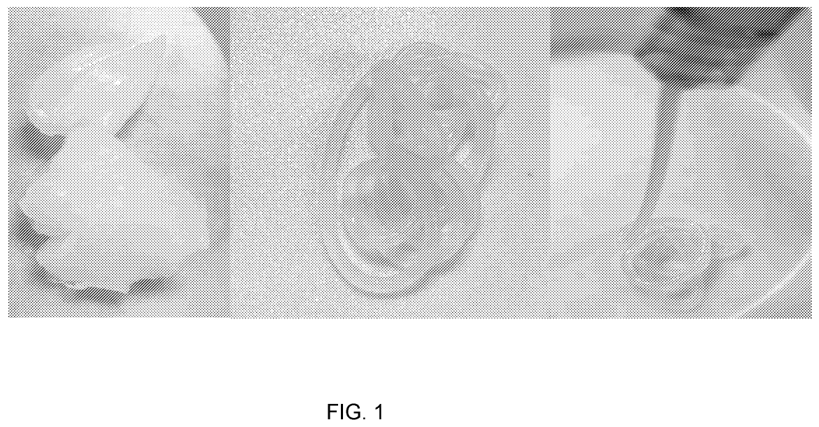

FIG. 2 depicts photographs of shear-thinning composition B.

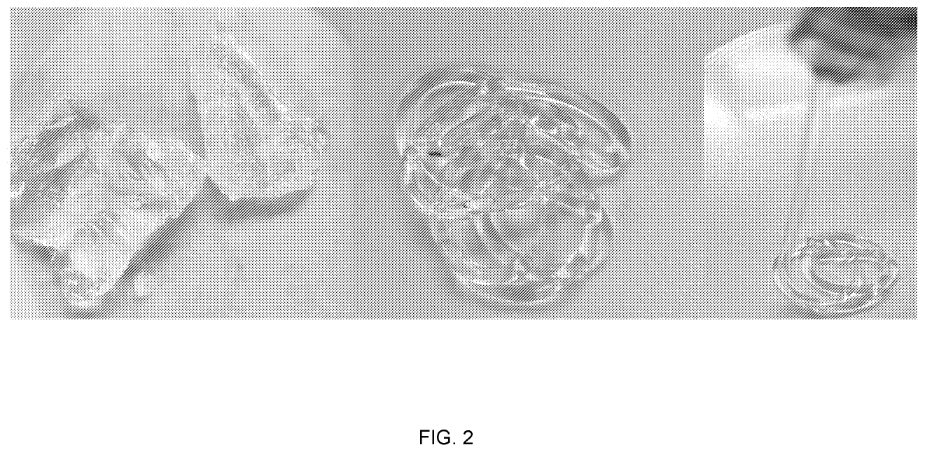

### DETAILED DESCRIPTION

### Definitions

While the following terms are believed to be well understood by one of ordinary skill in the art, the following definitions are set forth to facilitate explanation of the presently disclosed subject matter.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

The term “about” when immediately preceding a numerical value means a range of plus or minus an acceptable degree of variation in the art. In some embodiments, the term “about” encompasses 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “effective amount” and “therapeutically effective amount” are used interchangeably in this disclosure and refer to the amount of a compound or a therapeutically active agent that, when administered to a patient, is capable of performing the intended result. For example, an effective amount of a composition of the present disclosure is that amount which is required to treat a disease or medical condition disclosed herein. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The term “patient” or “subject” as used herein, includes humans and animals, preferably mammals. In some embodiments, the subject is a human. In some embodiments, the subject is suffering from or at risk of developing a lesion such as a carcinoma, a lymphoma, a blastoma, or a sarcoma.

Provided herein are compositions useful for ablation, sclerotherapy, and neurolytics, among other therapeutic applications. In some embodiments, the present disclosure provides compositions comprising ethanol, water, or a derivative thereof, and silicate nanoparticles. Methods of use, kits comprising the compositions, and a process of making the compositions are also provided.

As it relates to ablation and sclerotherapy, management of venous malformations includes compression, surgical resection, and obliteration of channel lumens by percutaneous sclerotherapy. Sclerosing agents, through direct vessel wall contact, cause endothelial damage, inflammation, and fibrosis that obliterate the vascular channels. Common sclerosing agents include ethanol, (foamed) polidocanol, sodium tetradecyl sulfate (STS), doxycycline, or bleomycin. Pure ethanol has the highest sclerosing power. However, standard sclerosing agents can cause non-target vessel necrosis and leakage. For a sclerosant to be effective, it must diffuse to its target tissue through a fluid medium and interact with the target tissue for a sufficient period of time to begin the process leading to sclerosis. Sclerosants are liquid, and diffusion from high to low concentrations will be on the order of centimeters per second (cm/s). Diffusion may be altered by turbulent flow such as that which occurs in a rapid flowing system, which may aid in mixing but carry away the agent. In contrast, slower laminar flow may allow traversal of the agent without mixing, making it entirely ineffective at the desired target. Either type of flow may lead to unintended embolization of liquid and even foam agents. The amount of flow together with permeability may also define a maximum agent concentration. This may be significant because it may alter the dose administered and target/non-target distribution.

In some embodiments, the present disclosure provides high viscosity compositions which can remain in place for increased contact time between the sclerosant and endothelium and permit the avoidance of leakage. Ethanol (EtOH) produces a long-lasting embolization effect by causing endothelial damage and thrombosis of the arteriolar lumen of tumor feeder vessels and tumor vasculature, thereby leading to infarction of the tumor. When injected into any vascular space (arterial, venous, lymphatic), ethanol denudes the endothelial cell from the vascular wall and precipitates its protoplasm. The most frequently described complication is colonic infarct caused by ethanol passing through the ventral area of the aorta to the inferior mesenteric artery. If a bolus of ethanol reaches the pulmonary vascular bed, pulmonary artery spasm can then occur. If the spasm becomes severe enough, it can lead to pulmonary hypertension and right heart failure, which causes decreased left heart filling and resultant systemic hypotension. Severe systemic hypotension causes decreased coronary artery perfusion. If severe enough, this can lead to cardiac arrhythmias such as electromechanical dissociation and asystole, or the absence of electrical activity in the heart. In addition, the speed of ethanol injection can also affect efficacy. Blood dilutes ethanol and therefore will weaken the effect of ethanol on tumor cells if it is injected slowly, yet rapid injection might destroy blood vessel walls and form a vessel-casting mold. Injection of too much mixture can lead to reflux into normal liver tissues or to the blood vessels that supply the gastrointestinal tract.

The compositions of the present disclosure can be used, in some embodiments, in place of absolute ethanol as an intravascular embolic agent. The gel formulation reduces the risk of inadvertent non-target ethanol embolization that may lead to devitalization of surrounding tissues.

Ethanol ablation is a form of cancer therapy where ethanol is injected directly into a tumor. It is currently used only for some types of liver and thyroid cancers and the treatment is notoriously limited because of the need to use large volumes of ethanol that can damage surrounding tissue. This means it is primarily only effective for tumors surrounded by a fibrous capsule that can contain the ethanol. Ethanol ablation and complete necrosis of tumors can be expected in tumors smaller than 3 cm, but the efficacy drastically decreases in larger tumors. While most commonly applied in the liver, ethanol ablation has also been successfully employed in treatment of cardiomyopathies, parathyroid and pancreatic tumors, adrenal metastases, and metastatic pelvic lymph nodes. Clinical use of liquid ablation agents, such as percutaneous ethanol injection, has declined in the past decade because of inefficient intratumoral diffusion, resulting in residual tumor and high recurrence rates. In addition, ethanol injection for ablation is associated with potentially serious complications, such as portal vein thrombosis, hemoperitoneum, liver failure, and death. Ethanol ablation continues to be used worldwide due to low-cost, lack of the need for specialized equipment, and wide availability.

In some embodiments, the compositions described herein overcome limitations of ethanol tumor ablation by using a gel composition that remains in the tumors. In some embodiments, the compositions of the present disclosure achieve reduced leakage of ethanol as compared to traditional liquid ethanol ablation. Without being bound by theory, the controlled viscosity of the alcoholic sclerosing compositions provided herein allows them to remain in contact in the tumor longer and causes accelerated dehydration and sclerosing of the cancer cells. In some embodiments, the compositions provided herein allow for drug loading and maximizing drug distribution and retention in the tumor.

Neurolysis refers to the deliberate destruction of a nerve or a network of interlacing nerves (i.e., a nerve plexus) with the aim of providing permanent relief from pain by interrupting the transmission of pain signals in the nerves. Nerve blocking refers to temporarily blocking the function of a nerve by injecting painkillers into an area around an affected nerve, thus blocking the transmission of pain signals, resulting in the temporary disabling of the nerve without causing permanent damage.

There are a number of techniques by which neurolysis may be performed. The most common method of causing permanent nerve destruction is the injection of a chemical such as alcohol. Alternately, an interventional radiologist may choose to use ablation techniques to destroy the nerves. In either case, in event with the interventional radiologist being able to insert a needle or a thermal probe locally so it is in contact with the nerve or the plexus, a subsequent injection of the chemical (e.g., ethanol) results in locally very high concentrations of the ablating agent, which can cause unwanted damage to neighboring tissues.

Accordingly, the present disclosure provides a composition for use in neurolysis that incorporates neurolytic agents, such as ethanol and phenol, into an injectable solid device. The injectable solid device can be delivered through percutaneous injection directly into a neural plexus or ganglion of interest or via endoscopic ultrasound-guided delivery. The compositions of the present disclosure provide a high viscosity material which can remain in place for increased contact time between the neurolytic agent and the neural plexus or ganglion, thus avoiding leakage and the need to provide high volumes and concentrations of neurolytic agent to the treatment site to achieve a therapeutic effect. The compositions of the present disclosure reduce the risk of inadvertent nontarget ethanol neurolysis that may lead to ablation of surrounding tissues. In certain embodiments, the compositions of the present disclosure can be used in place of absolute ethanol or phenol as a neurolytic agent.

### Compositions

In one aspect, provided herein is a composition for use in ablation or sclerotherapy, comprising an effective amount of an alcohol, silicate nanoparticles, and water, wherein the composition has a viscosity of about 2,000-15,000 mPa·s.

In one aspect, provided herein is a composition for use in ablation or sclerotherapy, comprising an effective amount of ethanol or a derivative thereof, silicate nanoparticles, and water, wherein the composition has a viscosity of about 2,000-15,000 mPa·s.

In one aspect, provided herein is a composition for use in ablation, comprising an effective amount of ethanol or a derivative thereof, silicate nanoparticles, and water, wherein the composition has a viscosity of about 2,000-15,000 mPa·s.

In one aspect, provided herein is a composition for use in sclerotherapy, comprising an effective amount of ethanol or a derivative thereof, silicate nanoparticles, and water, wherein the composition has a viscosity of about 2,000-15,000 mPa·s.

In some embodiments of the compositions provided herein, upon injection of the composition to a patient in need thereof, the implant stays in place for at least about 1 day. In some embodiments, the implant stays in place for at least about 5 days, at least about a week, at least about two weeks, at least about a month, at least about 60 days, at least about 90 days, at least about 180 days, at least about 1 year or more. In some embodiments, the implant stays in place for at least about 90 days.

In some embodiments of the compositions provided herein, upon injection of the composition to a patient in need thereof, the implant elutes the ethanol or a derivative thereof for at least about 1 day, at least about 2 days, at least about 4 days, at least about 5 days, at least about 7 days, or at least about 2 weeks, at least about a month, at least about 60 days, at least about 90 days, at least about 180 days, at least about 1 year, or more. In some embodiments of the compositions provided herein, upon injection to a patient in need thereof, the implant elutes the ethanol or a derivative thereof for at least 5 days. In some embodiments, ethanol is not released from the implant.

In some embodiments of the compositions provided herein, the ratio of silicate nanoparticles (e.g., Laponite) to ethanol or a derivative thereof is from about 1.0 to about 0.1 by weight, including about 1.0 by weight, about 0.9 by weight, about 0.8 by weight, about 0.7 by weight, about 0.6 by weight, about 0.5 by weight, about 0.4 by weight, about 0.3 by weight, about 0.2 by weight, to about 0.1 by weight, including all values and subranges therebetween.

In some embodiments, the compositions provided herein comprise about 1% to about 50% by weight of silicate nanoparticles (e.g., Laponite), including about 1% by weight, about 2% by weight, about 3% by weight, about 4% by weight, about 5% by weight, about 6% by weight, about 7% by weight, about 8% by weight, about 9% by weight, about 10% by weight, about 11% by weight, about 12% by weight, about 13% by weight, about 14% by weight, to about 15% by weight, about 16% by weight, about 17% by weight, about 18% by weight, about 19% by weight, about 20% by weight, about 21% by weight, about 22% a by weight, about 23% by weight, about 24% by weight, about 25% by weight, about 26% by weight, about 27% by weight, about 28% by weight, about 29% by weight, about 30% by weight, about 31% by weight, about 32% by weight, about 33% by weight, about 34% by weight, about 35% by weight, about 36% by weight, about 37% by weight, about 38% by weight, about 39% by weight, about 40% by weight, about 41% by weight, about 42% by weight, about 43% by weight, about 44% by weight, about 45% by weight, about 46% by weight, about 47% by weight, about 48% by weight, about 49% by weight, to about 50% by weight including all subranges and values therebetween. In some embodiments, the compositions provided herein comprise about 5% to about 15% by weight of silicate nanoparticles. In some embodiments, the silicate nanoparticle is a laponite disk.

In some embodiments of the compositions of the present disclosure, the ratio of water to ethanol or a derivative thereof ranges from about 10:90 by weight to about 100:0 by weight, including about 10:90 by weight, about 15:85 by weight, about 20:80 by weight, about 25:75 by weight, about 30:70 by weight, about 35:65 by weight, about 40:60 by weight, about 45:55 by weight, about 50:50 by weight, about 55:45 by weight, about 60:40 by weight, about 65:35 by weight, about 70:30 by weight, about 75:25 by weight, about 80:20 by weight, about 85:15 by weight, about 90:10 by weight, to about 100:0 by weight. In some embodiments of the compositions of the present disclosure, the ratio of water to ethanol or a derivative thereof is from about 100:0 to about 50:50 by weight.

In some embodiments of the compositions of the present disclosure, the ratio of silicate nanoparticles (e.g., Laponite) to ethanol or a derivative thereof is between about 90:10 by weight to about 10:90 by weight, including between about 90:10 by weight, about 85:15 by weight, about 80:20 by weight, about 75:25 by weight, about 70:30 by weight, about 65:35 by weight, about 60:40 by weight, about 55:45 by weight, about 50:50 by weight, about 45:55 by weight, about 40:60 by weight, about 35:65 by weight, about 30:70 by weight, about 25:75 by weight, about 20:80 by weight, about 15:85 by weight, to about 10:90 by weight including all values and subranges therebetween. In some embodiments, the ratio of silicate nanoparticles to ethanol or a derivative thereof is about 75:25 by weight. In some embodiments, the ratio of silicate nanoparticles to ethanol or a derivative thereof is about 60:40 by weight.

In aspects, the composition may be configured as a gel, the ablating agent (e.g. an alcohol, such as ethanol) may be present in about 5-80% w/w, including about 5% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, to about 80% w/w, including all values and subranges therebetween. In some embodiments, the silicate nanoparticles comprise silicate nanoplatelets. In some embodiments, the silicate nanoplatelets comprise a positively charged edge and a negatively charged surface. In some embodiments, the silicate nanoparticles are negatively charged.

In some embodiments, the silicate nanoparticles have a particle size from about 5 nm to about 500 nm, including about 5 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, to about 500 nm including all subranges and values and ranges therebetween. In some embodiments, the silicate nanoparticles are between about 25 nm to about 50 nm in diameter. In some embodiments, particle size is measured by Transmission Electron Microscopy (TEM).

In some embodiments, the average diameter of the silicate nanoparticles is about 5 nm to about 60 nm, including about 5 nm, about 10 nm, about 15 nm, about 20 nm, about 25 nm, about 30 nm, about 35 nm, about 40 nm, about 45 nm, about 50 nm, about 55 nm, to about 60 nm, including all values and subranges therebetween. In some embodiments, particle size is measured by Transmission Electron Microscopy (TEM).

In some embodiments, the average thickness of the silicate nanoparticles is about 0.5 nm to about 2 nm, including about 0.5 nm, about 0.6 nm, about 0.7 nm, about 0.8 nm, about 0.9 nm, about 1 nm, about 1.1 nm, about 1.2 nm, about 1.3 nm, about 1.4 nm, about 1.5 nm, about 1.6 nm, about 1.7 nm, about 1.8 nm, about 1.9 nm, to about 2.0 nm, including all subranges and values therebetween. In some embodiments, particle size is measured by Transmission Electron Microscopy (TEM).

In some embodiments, the composition may be formulated as a shear-thinning composition. Such compositions may be advantageous for delivery e.g., by injection of the composition to a target area through a delivery system in accordance with the methods provided herein, while retaining a high degree of stability after delivery to the target area. In some embodiments, as the quantity of silicate nanoparticles in the composition increases, the viscosity and mechanical properties increase.

In some embodiments, the viscosity of the compositions can be controlled to allow for delivery of the composition to the target area and to allow for retention of the composition at the target area. In some embodiments, the composition has a viscosity of about 2,000-15,000 mPa·s as measured by viscometry, including about 2,000 mPa·s, about 2,100 mPa·s, about 2,200 mPa·s, about 2,300 mPa·s, about 2,400 mPa·s, about 2,500 mPa·s, about 2,600 mPa·s, about 2,700 mPa·s, about 2,800 mPa·s, about 3,000 mPa·s, about 3,100 mPa·s, about 3,200 mPa·s, about 3,300 mPa·s, about 3,400 mPa·s, about 3,500 mPa·s, about 3,600 mPa·s, about 3,700 mPa·s, about 3,800 mPa·s, about 3,900 mPa·s, about 4,000 mPa·s, about 4,100 mPa·s, about 4,200 mPa·s, about 4,300 mPa·s, about 4,400 mPa·s, about 4,500 mPa·s, about 4,600 mPa·s, about 4,700 mPa·s, about 4,800 mPa·s, about 4,900 mPa·s, about 5,000 mPa·s, about 5,100 mPa·s, about 5,200 mPa·s, about 5,300 mPa·s, about 5,400 mPa·s, about 5,500 mPa·s, about 5,600 mPa·s, about 5,700 mPa·s, about 5,800 mPa·s, about 5,900 mPa·s, about 6,000 mPa·s, about 6,100 mPa·s, about 6,200 mPa·s, about 6,300 mPa·s, about 6,400 mPa·s, about 6,500 mPa·s, about 6,600 mPa·s, about 6,700 mPa·s, about 6,800 mPa·s, about 6,900 mPa·s, about 7,000 mPa·s, about 7,100 mPa·s, about 7,200 mPa·s, about 7,300 mPa·s, about 7,400 mPa·s, about 7,500 mPa·s, about 7,600 mPa·s, about 7,700 mPa·s, about 7,800 mPa·s, about 7,900 mPa·s, about 8,000 mPa·s, about 8,100 mPa·s, about 8,200mPa·s, about 8,300 mPa·s, about 8,400 mPa·s, about 8,500 mPa·s, about 8,600 mPa·s, about 8,700 mPa·s, about 8,800 mPa·s, about 8,900 mPa·s, about 9,000 mPa·s, about 9,100 mPa·s, about 9,200 mPa·s, about 9,300 mPa·s, about 9,400 mPa·s, about 9,500 mPa·s, about 9,600 mPa·s, about 9,700 mPa·s, about 9,800 mPa·s, about 9,900 mPa·s, about 10,000 mPa·s, about 10,100 mPa·s, about 10,200 mPa·s, about 10,300 mPa·s, about 10,400 mPa·s, about 10,500 mPa·s, about 10,600 mPa·s, about 10,700 mPa·s, about 10,800 mPa·s, about 10,900 mPa·s, about 11,000, mPa·s, about 11,100 mPa·s, about 11,200 mPa·s, about 11,300 mPa·s, about 11,400 mPa·s, about 11,500 mPa·s, about 11,600 mPa·s, about 11,700 mPa·s, about 11,800 mPa·s, about 11,900 mPa·s, about 12,000 mPa·s, about 12,100 mPa·s, about 12,200 mPa·s, about 12,300 mPa·s, about 12,400 mPa·s, about 12,500 mPa·s, about 12,600 mPa·s, about 12,700 mPa·s, about 12,800 mPa·s, about 12,900 mPa·s, about 13, 000 mPa·s, about 13,100 mPa·s, about 13,200 mPa·s, about 13,300 mPa·s, about 13,400 mPa·s, about 13,500 mPa·s, about 13,600 mPa·s, about 13,700 mPa·s, about 13,800 mPa·s, about 13,900 mPa·s, about 14,000 mPa·s, about14,100 mPa·s, about 14,200 mPa·s, about 14,300 mPa·s, about 14,400 mPa·s, about 14,500 mPa·s, about 14,600 mPa·s, about 14,700 mPa·s, about 14,800 mPa·s, about 14,900 mPa·s, to about 15,000 mPa·s, including all values and subranges therebetween.

In some embodiments, the yield stress of the composition may be configured such that the shear-thinning composition can flow freely through a delivery system upon application of a pressure greater than the yield stress, but flow substantially stops when the force applied is less than the force required to overcome the yield strength (e.g., upon delivery of the composition to the target site), forming a stiff gel state so as to be retained at the target site.

In some embodiments, the composition has a yield stress of from about 1 Pa to about 200 Pa, including from about 1 Pa, about 5 Pa, about 10 Pa, about 15 Pa, about 20 Pa, about 25 Pa, about 30 Pa, about 35 Pa, about 40 Pa, about 45 Pa, about 50 Pa, about 55 Pa, about 60 Pa, about 65 Pa, about 70 Pa, about 75 Pa, about 80 Pa, about 85 Pa, about 90 Pa, about 95 Pa, about 100 Pa, about 110 Pa, Pa, about 115 Pa, about 120 Pa, about 125 Pa, about 130 Pa, about 135 Pa, about 140 Pa, about 145 Pa, about 150 Pa, about 155 Pa, about 160 Pa, about 165 Pa, about 170 Pa, about 175 Pa, about 180 Pa, about 185 Pa, about 190 Pa, about 195 Pa, to about 200 Pa, including all values and subranges therebetween. In some embodiments, stress yield is measured by rheometry.

In some embodiments, the composition has a storage modulus (G′) range of 1,000-10,000 Pa as measured by rheometry, including about 1,000 Pa, about 1,500 Pa, about 2,000 Pa, about 2,500 Pa, about 3,000 Pa, about 3,500 Pa, about 4,000 Pa, about 4,500 Pa, about 5,000 Pa, about 5,500 Pa, about 6,000 Pa, about 6,500 Pa, about 7,000 Pa, about 7,500 Pa, about 8,000 Pa, about 8,500 Pa, about 9,000 Pa, about 9,500 Pa, to about 10,000 Pa, including all values and subranges therebetween. In some embodiments, the storage modulus is measured by rheometry.

In some embodiments, the composition has a loss modulus (G″) range of 100-1,000 Pa, including about 100 Pa, about 200 Pa, about 300 Pa, about 400 Pa, about 500 Pa, about 600 Pa, about 700 Pa, about 800 Pa, about 900 Pa, about 1,000 Pa, about 1,500 Pa, about 2,000 Pa, about 2,500 Pa, about 3,000 Pa, about 3,500 Pa, about 4,000 Pa, about 4,500 Pa, about 5,000 Pa, about 5,500 Pa, about 6,000 Pa, about 6,500 Pa, about 7,000 Pa, about 7,500 Pa, about 8,000 Pa, about 8,500 Pa, about 9,000 Pa, about 9,500 Pa, to about 10,000 Pa, including all values and subranges therebetween. In some embodiments, the loss modulus (G″) is measured by rheometry.

In some embodiments of the present disclosure, the composition is disposed within a delivery system (e.g., a vessel such as a catheter) selected for its ability to facilitate a user modulating one or more rheological properties of the composition (e.g. by applying manual pressure to a 5-FR general catheter, a 2.8 F catheter or a 2.4-Fr microcatheter). In some embodiments, the composition is injected via a 23 gauge needle. In some embodiments, the injection force applied is less than about 10 N. In some embodiments, the composition is injected via a 23 gauge needle, and the injection force applied is between about 0.1 Newtons (N) and about 10 N, including about 0.1 N, about 0.5 N, about 1 N, about 2 N, about 3 N, about 4 N, about 5 N, about 6 N, about 7 N, about 8 N, about 9 N, to about 10 N, including all values and subranges therebetween. In some embodiments, the composition is injected via a 2.8 F gauge catheter. In some embodiments, injection force applied is less than about 100N. In some embodiments, the composition is injected via a 2.8 F gauge catheter and the injection force applies is between about 1 N and about 100 N, including about 1 N, about 5 N, about 10 N, about 15 N, about 20 N, about 25 N, about 30 N, about 35 N, about 40 N, about 45 N, about 50 N, about 55 N, about 60 N, about 65 N, about 70 N, about 75 N, about 80 N, about 85 N, about 90 N, about 95 N, to about 100 N, including all values and subranges therebetween.

Advantageously, the administration of compositions of the present disclosure may result in e.g., retention of the composition in the site of injection, reduction of non-target migration of the agent, and reduction of local toxicity, compared to administration of the ablation agent alone.

In some embodiments, a further benefit is that lower doses of the embolic agent may be used, for example, such that the dosages of embolic agent may not only often be smaller, but also may be applied less frequently, or can be used in order to diminish the incidence of side-effects observed with embolic agent alone.

In some embodiments, the composition further comprises a contrast agent. The contrast agent allows visualization of embolic agent fluoroscopically. In some embodiments, the contrast agent is a fluorescent agent, for example a fluorescent dye such as fluorescein or indocyanine green, an iodine-based contrast agent, an MRI contrast agent or a CT contrast agent or a combination thereof. In some embodiments, the contrast agent is tantalum, iodine, or lipiodol. With detectable compound, the composition may be imaged to monitor the compositions distribution in vivo upon administration and/or during administration in real time.

In some embodiments, the composition further comprises a therapeutic agent selected from the group consisting of chemotherapeutic agents and sclerosing agents. In some embodiments, the therapeutic agent selected from the group consisting of acetic acid injectable agents, anesthetic agents, antibiotics, enzymes, biological agents, bioabsorbable polymers, biomaterials, conjugates, pharmaceutical drugs, genes, viruses, vasoconstricting agents, proteins, contrast agents, polymers, plant and animal tissue cell byproducts and derivatives, natural extracts/compounds and other biochemical agents. In some embodiments, the sclerosing agent is polidocanol, sodium tetradecyl sulfate (STS), doxycycline or bleomycin. In some embodiments, the composition includes a therapeutic agent selected from an anti-inflammatory agent, an embolic agent, and a chemotherapeutic agent. Illustrative embolic agents include, for example, stainless steel coils, absorbable gelatin pledgets and powders, polyvinyl alcohol foams, ethanol, glues and the like.

Anticancer agents used in combination with the compositions of the present disclosure may include agents selected from any of the classes known to those of ordinary skill in the art, including, for example, alkylating agents, anti-metabolites, plant alkaloids and terpenoids (e.g., taxanes), topoisomerase inhibitors, anti-tumor antibiotics, kinase inhibitors, hormonal therapies, molecular targeted agents, and the like. In some embodiments, the anticancer agent is one or more anticancer agents selected from the group consisting of Abraxane, Adriamycin, carboplatin, Cytoxan, daunorubicin, Doxil, Ellence, fluorouracil, Gemzar, Halaven, Ixempra, methotrexate, Mitomycin, mitoxantrone, Navelbine, Taxol, Taxotere, thiotepa, vincristine, and Xeloda.

In some embodiments, the composition further comprises one or more immunomodulatory agents useful for immunotherapy. In some embodiments, the immunotherapeutic agent in the composition is a monoclonal antibody, an immune effector cell, adoptive cell transfer, an immunotoxin, a vaccine, and/or a cytokine. In some embodiments, the immunotherapeutic agent is a cell-based immunotherapeutic agent, such as granulocyte colony-stimulating factor (G-CSF), interferons, imiquimod and cellular membrane fractions from bacteria, IL-2, IL-7, IL-12, various chemokines, synthetic cytosine phosphate-guanosine (CpG) oligodeoxynucleotides, and glucans. In some embodiments, the immunotherapeutic agent is an agent that induces immune checkpoint blockade, such as a checkpoint inhibitor (e.g., PD-1, CTLA-4 or PD-L1 checkpoint inhibitor). In some embodiments, the immunotherapeutic agent is one or more immunotherapeutic agents selected from the group consisting of Ipilimumab, Nivolumab, Pembrolizumab, Atezolizumab, Avelumab, and Durvalumab.

In some embodiments, the composition further comprises one or more polymers. Suitable polymers include synthetic polymers such as PNIPAM, PLGA, PLA, or PVA, or natural polymer such as gelatin, chitosan, or collagen and the like.

### Methods

The compositions of the present disclosure find use in any number of methods. For example, in some embodiments the present disclosure provides methods of treating a lesion, comprising the administering a therapeutically effective amount of a composition of the present disclosure to a patient in need thereof. In some embodiments, about 0.1 mL to 10 mL of a composition of the present disclosure is administered to a patient in need thereof, including about 0.1 mL, about 0.25 mL, about 0.5 mL, about 0.75 mL, about 1 mL, about 2 mL, about 3 mL, about 4 mL, about 5 mL, about 6 mL, about 7 mL, about 8 mL, about 9 mL, to about 10 mL, including all values and subranges therebetween.

In some embodiments, the present disclosure provides a method of reducing the size of a lesion in a subject in need thereof, comprising administering to the subject in need thereof a composition of the present disclosure.

In some embodiments, the lesion is a carcinoma, lymphoma, blastoma, or sarcoma. In some embodiments, the lesion is selected from a cancerous lesion, a precancerous lesion, a benign lesion, or growth in a subject thereof. In some embodiments, the lesion is a solid tumor. In some embodiments, the lesion is a non-capsulated tumor. In some embodiments, the lesion is a cyst, breast cancer, prostate cancer, colon cancer, squamous cell cancer, small-cell lung cancer, non-small cell lung cancer, ovarian cancer, cervical cancer, gastrointestinal cancer, pancreatic cancer, liver cancer, bladder cancer, hepatoma, colorectal cancer, uterine cervical cancer, endometrial carcinoma, salivary gland carcinoma, kidney cancer, vulval cancer, pancreatic cancer, thyroid cancer, hepatic carcinoma, skin cancer, melanoma, myeloma, head and neck cancer, Ewing sarcoma, epithelial tumor, or cervical cancer. In some embodiments, the present disclosure provides a method for treating cancer, or a precancerous lesion or benign lesion (such as a skin tag). In some embodiments, the lesion is an epithelial tumor or precancerous epithelial lesion.

In some embodiments the present disclosure provides methods of sclerotherapy, comprising the administering a therapeutically effective amount of a composition of the present disclosure to a patient in need thereof. In some embodiments, about 0.1 mL to 10 mL of a composition of the present disclosure is administered to a patient in need thereof, including about 0.1 mL, about 0.25 mL, about 0.5 mL, about 0.75 mL, about 1 mL, about 2 mL, about 3 mL, about 4 mL, about 5 mL, about 6 mL, about 7 mL, about 8 mL, about 9 mL, to about 10 mL, including all values and subranges therebetween.

In some embodiments, the compositions disclosed herein are used to form an embolism (e.g., intravascular embolism) in a target arca.

In some embodiments, the compositions of the present disclosure are useful for transarterial embolization ablation (TEA). In some embodiments, the compositions of the present disclosure are useful for combined embolization and therapeutic drug release (e.g., intravascularly) into a tumor.

In some embodiments, the composition is administered to treat a vascular malformation.

In an aspect, the present disclosure provides a method of visualizing (e.g., by fluoroscopy) the distribution of the composition with the aid of a contrast agent (e.g., a fluoroscopy contrast agent such as tantalum).

In the present methods, the administration of compositions provided herein comprises parenteral administration. Examples of the parenteral administration include but are not limited to intravenous (IV) administration, intraarterial administration, intramuscular administration, subcutaneous administration, intraosseous administration, intrathecal administration, or a combination thereof.

In some embodiments, the administration of compositions of the present disclosure is conducted via injection or infusion. In some embodiments, the injection is conducted via transvascular injection through a delivery system such as catheters (e.g., microcatheters) or via direct puncture and injection into the target area.

In some embodiments, the present disclosure provides kits for use in treating a lesion in a patient in need thereof. In some embodiments, the present disclosure provides kits for use in sclerotherapy in a patient in need thereof. In some embodiments, the kits of the present disclosure may comprise directions for administration. For example, the kit can include instructions to administer a composition of the present disclosure in a suitable manner to perform the methods described herein.

The kit can include one or more containers for compositions as described herein. In some embodiments, the kit contains separate containers, dividers or compartments for the composition and informational material. For example, the composition can be contained in a bottle, vial, or syringe. In some embodiments, the separate elements of the kit are contained within a single, undivided container. For example, the composition is contained in a bottle, vial, or syringe that has attached thereto the informational material in the form of a label. In some embodiments, the kit includes a plurality (e.g., a pack) of individual containers, each containing one or more unit dosage forms of a composition described herein. For example, the kit can include a plurality of syringes each equipped with a needle, and each containing a single unit dose of a composition described herein, and infusion set. The containers of the kits can be air tight, waterproof (e.g., impermeable to changes in moisture or evaporation), and/or light-tight.

### Neurolysis

Neurolysis is a technique used in pain management where neurolytic agents are used to induce temporary or permanent degeneration of nerve fibers related to pain transmission. Chemical neurolytics, including ethanol and phenol, can be delivered to a group of nerves, called a plexus or a ganglion, to block the pain to a specific organ or region of the body.

Because of the low viscosity and high dispersity of alcohol in tissue, current applications of this technique involve injections of large volumes of ethanol to a nerve ganglion of interest to insure therapeutic effect. Because this large volume of ethanol injection is also associated with patient pain, the development of a localized method of neurolysis with longer retention is needed.

Accordingly, the present disclosure provides a composition for use in neurolysis by incorporating neurolytic agents, including ethanol and phenol, into an injectable solid device. The injectable solid device can be delivered through percutaneous injection directly into the neural plexus or ganglion of interest or via endoscopic ultrasound-guided delivery (EUS). The injectable solid device allows for stable delivery of neurolytic agents at the site of interest, minimizing the effects of neurolytic agent diffusion away from the delivery site. In some embodiments, the present disclosure provides high viscosity compositions which can remain in place for increased contact time between the neurolytic agent and the neural plexus or ganglion and avoid leakage. The compositions of the present disclosure can be used, in some embodiments, in place of absolute ethanol or phenol as a neurolytic agent. The gel formulation reduces the risk of inadvertent nontarget ethanol neurolysis that may lead to ablation of surrounding tissues.

### Sprayable Gel Formation

Surgical resection remains the cornerstone of treatment for a number of primary malignant cancers. Despite the prevalence of this technique in the clinic, cancer recurrence as a result of residual tumor infiltration and circulating tumor cells is still an issue. Surgical resection involves the removal of a primary tumor with the goal of complete eradication of cancer. Unfortunately, this ideal is not always achieved as a positive surgical margin can occur where cancer cells are present at the edge of the resection area. For common primary tumors, positive surgical margins occur for between about 5% and 35% of treated patients. In cases where positive surgical margins occur, adjuvant therapies including chemotherapy and radiotherapy are required, which can carry significant financial and prognostic implications.

In areas where full surgical resection cannot be performed due to risk of tissue damage or nearby vital organs, debulking is performed. Debulking is a process in which as much of the tumor as possible is removed to make chemotherapy or radiation more effective. Inherently, this technique leaves remaining tumor cells at the surgical site, and thus leaves the opportunity for recurrence. Accordingly, there remains a need to locally ablate lingering tumor cells at the time of resection surgery to prevent regrowth of primary tumors or metastases thereof to other regions.

In embodiments, the present disclosure provides a composition for use in tumor ablation by incorporating ablative agents, including ethanol, into a sprayable solid device. The sprayable solid device can be used, in some embodiments, proactively at the time of surgical resection. The sprayable solid device may be further used after the primary tumor resection or debulking is performed in a separate procedure and after demonstration of a positive surgical margin. In embodiments, the sprayable solid device serves as a space filling material after surgical resection or debulking. In some embodiments, the compositions of the present disclosure can be used in place of adjuvant therapies, such as chemotherapy or radiotherapy. In some embodiments, the compositions of the present disclosure can be used in tandem with adjuvant therapies, such as chemotherapeutics or radiotherapy.

### EXAMPLES

Example 1: Preparation of sclerosing shear-thinning compositions The compositions were prepared by dispersing silicate nanoplatelets laponite in water/ethanol solutions. Sterile water (4° C.) was used to allow for full dispersion and exfoliation of nanoplatelet particles prior to gelling. The nanocomposite compositions were made by speed mixing at 3000 rpm for 5 min and this process was repeated three times. Nanoplatelet gels were allowed to sit at room temperature to fully hydrate. The composition gel was transferred to a 50 ml master syringe and mixed at 3000 rpm for 5 min. Using the master syringes with a female luer connector, the final 1 cc or 3 cc COP syringes were filled and cured at 4C° refrigerator.

### Composition A

Shear-thinning composition A was prepared by mixing laponite (9%; 3.6 g/40 g solution of water-alcohol) with a water-alcohol solution (60%-40% w/w).

### Composition B

Shear-thinning composition A was prepared by mixing laponite (9%; 3.6 g/54 g solution of water-alcohol) with a water-alcohol solution (74%-26% w/w).

1. A composition for use in ablation or sclerotherapy, comprising
an effective amount of ethanol or a derivative thereof,
silicate nanoparticles,
and water,
wherein the composition has a viscosity of about 2,000-15,000 mPa·s.

2. The composition of claim 1, wherein upon injection to a patient in need thereof, the composition provides an implant that stays in place for at least 90 days.

3. The composition of claim 1, wherein upon injection to a patient in need thereof, the composition provides an implant that elutes the ethanol or a derivative thereof for at least 90 days.

4. The composition of claim 1, wherein the ratio of silicate nanoparticles to ethanol or a derivative thereof is from about 1.0 to about 0.1 by weight.

5. The composition of claim 1, wherein the composition comprises about 5% to about 15% by weight of silicate nanoparticles.

6. The composition of claim 1, wherein the composition comprises about 10% by weight of silicate nanoparticles.

7. The composition of claim 1, wherein the ratio of water to ethanol or a derivative thereof is from about 100:0 to about 50:50 by weight.

8. The composition of claim 1, wherein the ratio of silicate nanoparticles to ethanol or a derivative thereof is 75:25 by weight.

9. The composition of claim 1, wherein the ratio of silicate nanoparticles to ethanol or a derivative thereof is 60:40 by weight.

10. The composition of claim 1, wherein the silicate nanoparticles comprise silicate nanoplatelets.

11. The composition of claim 10, wherein the silicate nanoplatelets comprise a positively charged edge and a negatively charged surface.

12. The composition of claim 1, wherein the silicate nanoparticles are negatively charged.

13. The composition of claim 1, wherein the average diameter of the silicate nanoparticles is about 5 nm to about 60 nm.

14. The composition of claim 1, wherein the average thickness of the silicate nanoparticles is about 0.5 nm to about 2 nm.

15. The composition of claim 1, wherein the composition has a yield stress of from about 1 Pa to about 200 Pa.

16. The composition of claim 1, wherein the composition is a shear-thinning composition.

17. The composition of claim 1, wherein the composition further comprises a contrast agent.

18. The composition of claim 1, wherein the composition further comprises a therapeutic agent selected from the group consisting of chemotherapeutic agents and sclerosing agents.

19. The composition of claim 1, wherein the composition further comprises a therapeutic agent selected from the group consisting of acetic acid injectable agents, anesthetic agents, antibiotics, enzymes, biological agents, bioabsorbable polymers, biomaterials, conjugates, pharmaceutical drugs, genes, viruses, vasoconstricting agents, proteins, plant and animal tissue cell byproducts and derivatives, natural extracts/compounds and other biochemical agents.

20. A method of treating a lesion, comprising administering a therapeutically effective amount of the composition of claim 1 to a patient in need thereof.

21. The method of claim 20, wherein the lesion is a carcinoma, lymphoma, blastoma, or sarcoma.

22. The method of claim 20, wherein the lesion is a cyst, breast cancer, prostate cancer, colon cancer, squamous cell cancer, small-cell lung cancer, non-small cell lung cancer, ovarian cancer, cervical cancer, gastrointestinal cancer, pancreatic cancer, liver cancer, bladder cancer, hepatoma, colorectal cancer, uterine cervical cancer, endometrial carcinoma, salivary gland carcinoma, kidney cancer, vulval cancer, pancreatic cancer, thyroid cancer, hepatic carcinoma, skin cancer, melanoma, myeloma, head and neck cancer, Ewing sarcoma, epithelial tumor, or cervical cancer.

23. A method of sclerotherapy, comprising administering a therapeutically effective amount of the composition of claim 1 to a patient in need thereof.

24. The method of claim 23, wherein the composition is administered to treat a vascular malformation.

25. A composition for use in neurolysis, comprising an effective amount of a neurolytic agent (such as ethanol or phenol), silicate nanoparticles, and water.

26. The composition of claim 25, wherein upon injection to a patient in need thereof, the composition provides an implant that stays in place for at least 1 day.

27. The composition any claim 25, wherein the composition further comprises one or more polymers selected from the group consisting of PNIPAM, PLGA, PLA, PEG, PVA, gelatin, chitosan, and collagen, or a mixture thereof.

28. The composition of claim 25, wherein upon injection to a patient in need thereof, the composition provides an implant that elutes the neurolytic agent.

29. The composition of claim 25, wherein the composition further comprises a contrast agent.

30. A method of pain management, comprising administering a therapeutically effective amount of the composition of claim 25 to a patient in need thereof.

31. The method of claim 30, wherein the method of pain management is for palliative care in cancer treatment.

32. The method of claim 30, wherein the method of pain management is for ischemic rest pain.

33. The method of claim 30, wherein the composition is used for renal denervation.

34. The method of claim 30, wherein the composition is used for genicular nerve degeneration.

35. A composition for ablation of residual tumor cells post-surgical resection, comprising an effective amount of an ablative agent (such as ethanol), silicate nanoparticles, and water.

36. The composition of claim 35, wherein the composition is delivered via spraying.

37. The composition of claim 35, wherein the composition further comprises a contrast agent.

38. The composition of claim 35, wherein the composition further comprises one or more polymers including at least one of PNIPAM, PLGA, PLA, PVA, gelatin, chitosan, and collagen.

39. A method of tumor therapy, comprising administering a therapeutically effective amount of the composition of claim 35 to a patient in need thereof.

40. The method of claim 39, wherein the tumor therapy is applied following full surgical resection.

41. The method of claim 39, wherein the tumor therapy is applied proactively at the time of full surgical resection.

42. The method of claim 39, wherein the tumor therapy is applied in the case of positive surgical margins.

43. The method of claim 39, wherein the composition ablates tumor margins.

44. The method of claim 39, wherein the tumor therapy is applied proactively at the time of debulking.

45. The method of claim 39, wherein the composition further serves as a space-filling material after surgical resection or debulking.

46. The composition of claim 39, wherein the composition further comprises a therapeutic agent selected from the group consisting of chemotherapeutic agents and immune oncology agents.
